(19) United States
(12) Patent Application Publication
(10) Pub. No.: US 2025/0331802 A1
(43) Pub. Date: Oct. 30, 2025

(54) NOISE VARIANCE STABILIZATION IN X-RAY IMAGING

(71) Applicant: Siemens Healthineers AG, Forchheim (DE)

(72) Inventors: Martino Leghissa, Wiesenthau (DE); Sai Gokul Hariharan, Forchheim (DE); Heiko Rohdjeß, Grossenseebach (DE)

(21) Appl. No.: 19/188,312

(22) Filed: Apr. 24, 2025

(30) Foreign Application Priority Data
Apr. 24, 2024 (EP) 24172211

(51) Int. Cl. A61B 6/00; A61B 6/06; A61B 6/40; A61B 6/46; A61B 6/58; G06N 20/00; G06T 5/60; G06T 5/70; G06T 7/00

(52) U.S. Cl. CPC A61B 6/5282; A61B 6/06; A61B 6/4035; A61B 6/461; A61B 6/547; A61B 6/585; G06N 20/00; G06T 5/60; G06T 5/70; G06T 7/0002; G06T 2207/10116; G06T 2207/20081; G06T 2207/20084

(57) ABSTRACT

For generating a noise-variance-stabilized X-ray image, an X-ray image generated by an X-ray imaging system and a corresponding set of imaging parameters of the X-ray imaging system are received. A noise level parameter is computed by simulating an energy deposition of X-ray quanta emitted by an X-ray source of the X-ray imaging system on an X-ray detector of the X-ray imaging system depending on the set of imaging parameters. The noise-variance-stabilized X-ray image is generated by applying a variance-stabilizing transformation that depends on the noise level parameter, to the X-ray image.

This application claims the benefit of European Patent Application No. EP 24172211, filed on Apr. 24, 2024, which is hereby incorporated by reference in its entirety.

### BACKGROUND

The present embodiments relate to generating a noise-variance-stabilized X-ray image, and to processing an X-ray image.

In X-ray imaging, noise content in two-dimensional (2D) images depends on various parameters. One typical measure of image quality is the signal-to-noise ratio (SNR), which is determined as a ratio of a signal y in an image that is, for example, expressed in digital units or gray values, and is provided by the X-ray detector, to the noise standard deviation σ. It is well known that SNR=y/σ depends on the X-ray dose; more specifically, SNR scales typically with square root of the X-ray dose. However, improving the image quality by increasing the dose is limited by the ALARA (“As Low As Reasonably Achievable”) principle, which basically requires to minimize or limit the X-ray dose.

A dose-independent quantity to characterize the noise content in X-ray images is the variance-to-signal ratio σ²/y. To be more precise, in flat panel X-ray imaging, the total noise variance may depend on various contributions (e.g., σ²=σ_{q}²+σₙ²), with the quantum noise contribution σ_{q}², which is related to the radiation field and is also referred to as Poisson noise, since it may be modelled using a Poisson distribution, and the electronic noise σₙ² of the X-ray detector, also referred as Gaussian noise, since it may be modelled using a Gaussian distribution.

Today's X-ray image processing pipelines are to work with the different noise characteristics (e.g., different mixtures of Poisson and Gaussian noise) that may occur depending on various parameters of the imaging scenario. The different contributions depend on different imaging parameters of the X-ray imaging system, such as exposure parameters, geometry parameters, the imaged object, and so forth. Therefore, image processing algorithms, specially denoising algorithms but also algorithms for edge enhancement, contrast enhancement, segmentation, etc., are to take into consideration all different noise characteristics. This may require that different variants of the image processing algorithms or parameterizations are provided for different regimes of the noise characteristics.

This problem is overcome by applying a variance-stabilizing transformation to the X-ray image, which stabilizes or normalizes the noise variance, for example, to a known constant, such that the image processing pipeline is applied to always the same normalized noise characteristics. The general Anscombe transform (GAT) is such a variance-stabilizing transformation.

In the publication S. Hariharan et al.: “Learning-based X-ray Image Denoising Utilizing Model-based Image Simulations,” in Shen, D., et al.: “Medical Image Computing and Computer Assisted Intervention—MICCAI 2019,” MICCAI 2019, Lecture Notes in Computer Science, vol. 11769, Springer, Cham, the generalized Anscombe transform is applied to medical X-ray imaging. Further, it is described how a trained artificial neural network may be used as a denoising algorithm based on accordingly transformed X-ray images.

The usage of the noise variance-stabilization comes along with the challenge, that for applying the transformation, knowledge of noise parameters is needed, especially knowledge of a noise level parameter α=(σ²−σₙ²)/yₘ and the electronic noise σₙ², where yₘ corresponds to the mean signal. While σₙ² may be obtained by calibrating the X-ray detector, a may be obtained in a data-driven approach by analyzing the input X-ray images as described in the publication S. Hariharan et al.: “Data-driven estimation of noise variance stabilization parameters for low-dose x-ray images,” 2020, Phys. Med. Biol., 65, 225027. However, this requires additional time-critical computational efforts, which is a draw back not only but especially for real-time fluoroscopy applications. Further, purely data-driven approaches may suffer from stability problems in scenarios where the X-ray images exhibit a large degree of structure or intensity variation.

The publications I. Cunningham et al: “A spatial-frequency dependent quantum accounting diagram and detective quantum efficiency model of signal and noise propagation in cascaded imaging systems,” 1994, Med. Phys., 21:417-427 and J. Siewerdsen et. al: “Empirical and theoretical investigation of the noise performance of indirect detection, active matrix flat-panel imagers (AMFPIs) for diagnostic radiology,” 1997, Med Phys., 24(1), 71-89, describe analytical models of imaging systems.

The publication J. Punnoose et al.: “Technical Note: spektr 3.0—A computational tool for x-ray spectrum modeling and analysis,” Med Phys., 2016, 43 (8), 4711 describes a computational toolkit, denoted as spektr 3.0, to calculate X-ray spectra based on a tungsten anode spectral model using an interpolating cubic splines algorithm (TASMICS).

The publication S. Agostinelli et al.: “Geant4—a simulation toolkit,” Nuclear Instruments and Methods in Physics Research Section A: Accelerators, Spectrometers, Detectors and Associated Equipment, 506 (3): 250 describes Geant4, a platform for simulating the passage of particles through matter using Monte-Carlo methods.

In the publication by O. Ronneberger et al.: “U-Net: Convolutional Networks for Biomedical Image Segmentation” (arXiv:1505.04597), the U-Net architecture is described, a widely used CNN architecture for image segmentation, which may, however, also be used for other computer vision tasks.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the draw backs of data-driven approaches when computing a noise-variance-stabilized X-ray image are overcome or reduced. For example, the robustness may be increased, and/or the computational effort may be reduced.

The present embodiments are based on the idea of determining a noise level parameter by simulating an energy deposition of X-ray quanta on the X-ray detector.

According to an aspect of the present embodiments, a computer-implemented method for generating a noise-variance-stabilized X-ray image is provided. Therein, an X-ray image that is or has been generated by an X-ray imaging system, and a corresponding set of imaging parameters of the X-ray imaging system are received. A noise level parameter is computed by simulating an energy deposition of X-ray quanta emitted by an X-ray source of the X-ray imaging system on an X-ray detector of the X-ray imaging system depending on the set of imaging parameters. The noise-variance-stabilized X-ray image is generated by applying a variance-stabilizing transformation, that depends on the noise level parameter, to the X-ray image.

Unless stated otherwise, all acts of the computer-implemented method may be performed by a data processing system that includes at least one data processing device. For example, the at least one data processing device is configured or adapted to perform the acts of the computer-implemented method. For this purpose, the at least one data processing device may, for example, store a computer program including instructions that, when executed by the at least one data processing device, cause the at least one data processing device to execute the computer-implemented method. The expressions “data processing system” and “at least one data processing device” may be used interchangeably here and in the following. This holds also for respective expressions derived therefrom.

In case the at least one data processing device includes two or more data processing devices, certain acts carried out by the at least one data processing device may also be understood such that different data processing devices carry out different acts or different parts of an act. For example, it is not required that each data processing device carries out the acts completely. In other words, carrying out the acts may be distributed amongst the two or more data processing devices.

From each implementation of the computer-implemented method, a respective implementation of a method for generating a noise-variance-stabilized X-ray image, which is not purely computer-implemented, is obtained by including respective acts of generating the X-ray image by the X-ray imaging system.

The X-ray image is, for example, a two-dimensional X-ray protection image that displays an object or a part of the object (e.g., a patient or a body part of the patient) that is, for example, located on a patient table of the X-ray imaging system. This object or the part of the object is denoted as image object in the following.

The set of imaging parameters is given by a respective set of imaging parameters that have been used or were present or were applied when generating the X-ray image using the X-ray imaging system. The set of imaging parameters may include, for example, any parameters or conditions that affect or potentially affect the noise content (e.g., the quantum noise present) in the X-ray image. This may include exposure parameters, geometrical parameters, detector parameters of the X-ray detector, parameters of the imaged object, and so forth.

The noise level parameter α is, for example, a parameter of the noise level function, NLF, of the imaging process, which depends on quantum noise. For example, denoting the variance of the total noise by σ²=σ_{q}²+σₑ², where σ_{q}² denotes the variance of the quantum noise and σₑ² denotes the variance of the electronic noise, the NLF may be denoted by σ²=α·yₘ+σₑ², with α=(σ²−σₑ²)/yₘ. Therein, yₘ corresponds to the mean signal in the resulting X-ray image.

The variance-stabilizing transformation depends parametrically on the noise level parameter α. Therefore, determining α is required in order to apply the variance-stabilizing transformation to the X-ray image. According to the present embodiments, this is achieved by simulating the energy deposition. This may be understood such that computing the noise level parameter includes simulating the energy deposition. The energy deposition may, for example, be quantified and, for example, simulated as ratio of the, within a detector pixel, squared deposited energy's mean to the mean of the deposited energy, sometimes denoted as E2E-value: E̅²̅/Ē. The noise level parameter is then computed depending on the E2E-value (e.g., based on calibration measurements assuming a linear relation between the noise level parameter and the E2E-value: α=α+b·E̅²̅/Ē).

Simulating the energy deposition may involve one or more simulation steps that simulate respective physical processes occurring in the complete imaging path from generating the X-rays by the X-ray source, the X-rays being transmitted, scattered, absorbed, and/or attenuated by the imaged object and/or other objects in the beam path, and/or further processes occurring until a part of the X-rays reaches the surface of the X-ray detector. In other words, one or more physically motivated models are used in order to compute the noise level parameter, in contrast to using a data-driven approach or image analysis. Further, also, other physical processes involved in the conversion of the deposited energy into respective detector signals of the X-ray detector and eventually the generation of the X-ray image in terms of image pixels may be modeled or simulated. In addition, physical processes involved in the generation of detector signals, also image processing algorithms in the pipeline may have an impact on the NLF. Therefore, the model for the NLF may consider such algorithmic processes.

The noise-variance-stabilized X-ray image may then be used as a basis for various known X-ray image processing algorithms, such as denoising algorithms, edge enhancement algorithms, contrast enhancement algorithms, medical segmentation algorithms, and so forth. For example, the noise-variance-stabilized X-ray image may be used for the algorithms instead of the X-ray image itself, which has the advantage that the input to the respective algorithm always has a consistent noise variance irrespective of the imaging parameters used for generating the respective X-ray image.

According to a number of implementations, a variance of an electronic noise of the X-ray detector is received, and the variance stabilizing transformation is carried out depending on the variance of the electronic noise. For example, the variance stabilizing transformation depends parametrically on the variance of the electronic noise.

In this way, the reliability of the variance stabilization is improved. The variance of the electronic noise may, for example, be obtained from calibration measurements of the X-ray detector. It may, for example, be considered as a given parameter of the X-ray detector and, for example, its value does not need to be determined during the computer-implemented method according to the present embodiments.

According to a number of implementations, required transformation parameters for all relevant different scenarios are precalculated and derived at runtime (e.g., using a lookup table). According to a number of (e.g., several) implementations, the precalculated transformation parameters are parametrized as a function of the imaging parameters.

According to a number of implementations, the noise level parameter is computed depending on the simulated energy deposition by using a detector model for the X-ray detector that models a conversion of X-ray quanta to optical photons and a conversion of the optical photons to an electrical detector signal (e.g., a digital electrical detector signal and, for example, a construction of respective pixel values of the X-ray image depending on the electrical detector signal).

In this way, the accuracy of the determined noise level parameter is further increased. Respective detector models for X-ray detectors are known (e.g., cascaded linear system models). For example, detector models according to the publications of Cunningham et al. or Siewerdsen et al. mentioned in the Background of the present disclosure may be used.

According to such models, the noise level parameter may, for example, be computed as

$$α = b \cdot ( {c}_{0} + {c}_{1} \overline{\frac{{E}^{2}}{\overline{E}}} ) ,$$

The parameters are determined by fitting respective experimental calibration data using the X-ray imaging system and, for example, phantom objects to obtain calibration data for various different sets of imaging parameters. However, alternatively or in addition, real patient measurements may be used.

According to a number of implementations, the set of imaging parameters includes exposure parameters of the X-ray source (e.g., a peak kilovoltage of the X-ray source used for generating the X-ray image and/or a tube current of the X-ray source used for generating the X-ray image and/or an X-ray pulse duration used for generating the X-ray image).

Exposure parameters such as this are known to have a significant impact on the energy spectrum of the X-ray quanta emitted by the X-ray source. Consequently, such exposure parameters also affect the noise content in the X-ray image and, for example, the variance of the quantum noise and the noise level parameter significantly. Simulating the energy deposition depending on the exposure parameters therefore allows to determine the noise level parameter with increased accuracy and, consequently, makes the variance stabilization more effective.

According to a number of implementations, the set of imaging parameters includes filter properties of an X-ray filter of the X-ray imaging system (e.g., a filter material and/or a filter thickness of the X-ray filter).

The X-ray filter is, for example, arranged between the X-ray source and the imaged object. The X-ray filter and the X-ray source may, for example, be part of a source unit of the X-ray imaging system (e.g., of a C-arm of the X-ray imaging system). The choice of the filter material may, for example, depend on an anode material of the X-ray source and on the targeted amount of beam hardening. A common choice for tungsten anodes is copper filters. However, others filter materials may include nickel, aluminum, or other metals.

The filter material as well as the filter thickness have a significant impact on the energy spectrum of the X-ray quanta after passing the X-ray filter. Consequently, the filter material and the filter thickness also affect the noise content in the X-ray image and, for example, the variance of the quantum noise and the noise level parameter significantly. Simulating the energy deposition depending on the filter material and/or the filter thickness therefore allows to determine the noise level parameter with increased accuracy and, consequently, makes the variance stabilization more effective.

According to a number of implementations, the set of imaging parameters includes collimator properties of an X-ray collimator of the X-ray imaging system (e.g., a collimator opening size of the X-ray collimator).

The X-ray collimator is, for example, arranged between the X-ray filter and the imaged object. The X-ray collimator may, for example, be part of the source unit of the X-ray imaging system. The choice of the collimator opening size may, for example, depend on the part of the object, which is to be imaged.

The collimator opening size has a significant impact, for example, on the scattering of X-ray quanta at the collimator and, since the scattering is energy dependent, also on the energy spectrum of the X-ray quanta after passing the X-ray filter. Consequently, the collimator opening size also affects the noise content in the X-ray image and, for example, the variance of the quantum noise and the noise level parameter significantly. Simulating the energy deposition depending on the collimator properties therefore allows to determine the noise level parameter with increased accuracy and, consequently, makes the variance stabilization more effective.

According to a number of implementations, the set of imaging parameters includes a zoom factor used for generating the X-ray image.

The zoom factor corresponds to a part of a detector array of the X-ray detector having data that is used for generating the X-ray image, that may be smaller than the full detector array. While the zoom factor may have a smaller effect on the noise content of the X-ray image than the exposure parameters, the filter properties, and the collimator properties, the effect may not be neglectable in some applications. Simulating the energy deposition depending on the zoom factor may therefore make the variance stabilization more effective.

According to a number of implementations, the set of imaging parameters includes geometrical parameters of the X-ray imaging system and the imaged object, respectively.

The geometrical parameters may, for example, include a position and/or an orientation of the X-ray source with respect to the imaged object and/or a position and/or orientation of the X-ray detector with respect to the imaged object. For example, in case the X-ray imaging system includes a C-arm carrying the X-ray source and the X-ray detector, the geometrical parameters may include a position and/or an angulation (e.g., an angular position and an orbital position) of the C-arm.

The geometrical parameters may, for example, include, in some implementations, a position and/or an orientation of a patient table, on which the imaged object is located, or a part of the patient table.

The geometrical parameters may also include an effective thickness of the imaged object (e.g., in terms of a water equivalent thickness (WET)). The geometrical parameters may also include an effective thickness of another object in the beam path between the X-ray source and the X-ray detector (e.g., the anti-scattering grid, a further anti-scattering grid, and/or a spectral filter).

The geometrical parameters have a significant impact, for example, on the scattering and transmission and, for example, absorption of X-ray quanta by the imaged object and, in some cases, further objects in the beam path. Consequently, the geometrical parameters also affect the noise content in the X-ray image and, for example, the variance of the quantum noise and the noise level parameter significantly. Simulating the energy deposition depending on the geometrical parameters therefore allows to determine the noise level parameter with increased accuracy and, consequently, makes the variance stabilization more effective.

According to a number of implementations, the set of imaging parameters includes a gain factor of the X-ray detector and/or a status parameter of an anti-scattering grid of the X-ray imaging system and/or a pixel binning parameter of the X-ray detector. The status parameter of the anti-scattering grid may, for example, be whether the anti-scattering grid is in the beam path or not.

The gain factor, the pixel binning parameter, and the presence of the anti-scattering grid in the beam path have a significant impact, for example, on the noise content in the X-ray image and, for example, the variance of the quantum noise and the noise level parameter. Simulating the energy deposition depending on the gain factor and/or the status parameter of an anti-scattering grid and/or the pixel binning parameter therefore allows to determine the noise level parameter with increased accuracy and, consequently, makes the variance stabilization more effective.

According to a number of implementations, the set of imaging parameters includes a status parameter of a further removable and/or configurable part in the beam path that affects X-ray absorption. The explanations with regard to the anti-scattering grid hold analogously.

According to a number of implementations, the variance-stabilizing transformation is a generalized Anscombe transformation.

The generalized Anscombe transformation is a well-known variance-stabilizing transformation that is also applicable in the context of X-ray images, and has proven particularly suitable for mixtures of Poisson and Gaussian noise. Consequently, a particularly reliable variance stabilization is achieved. The generalized Anscombe transformation is, for example, given by

$${y}^{′} = \frac{2}{α} \sqrt{α \cdot y + \frac{3}{8} {α}^{2} + {σ}_{n}^{2}} ,$$

where y denotes a pixel value of the X-ray image, y′ denotes the respective pixel value of the noise-variance-stabilized X-ray image, and σₙ² denotes the variance of the electronic noise of the X-ray detector, which is predetermined, for example, during a calibration phase. α denotes the noise level parameter computed by simulating the energy deposition as described.

Alternatively, other variance-stabilizing transformations of the form

$${y}^{′} = f ( α ) \sqrt{h \cdot α + g ( α , {σ}_{n}^{2} )} ,$$

may be used, where f and g denote functions of α and (α, σₙ²), respectively, and h is a constant.

According to a number of implementations, a Monte Carlo model of the X-ray imaging system and/or an analytical model of the X-ray imaging system is used to simulate the energy deposition.

For the Monte Carlo model, for example, Geant4 may be used. The analytical model may, for example, be based on the formalism of Cunningham et al. mentioned above.

In this way, a particularly accurate simulation of the energy deposition and, for some implementations, further processes happening in the X-ray detector, may be achieved.

According to a number of implementations, the simulation of the energy deposition is performed offline, and the result of the simulation is stored in a look-up-table, which is read-out in real-time.

According to a number of implementations, simulating the energy deposition includes simulating a collimation (e.g., of X-ray beams or X-ray quanta) by an X-ray collimator of the X-ray imaging system (e.g., using one or more Monte Carlo simulations).

Consequently, a particularly accurate simulation of the energy deposition is achieved.

According to a number of implementations, simulating the energy deposition includes simulating an X-ray attenuation and/or X-ray scattering and/or X-ray transmission by the imaged object and/or by one or more further objects in the beam path (e.g., using one or more Monte Carlo simulations).

Consequently, a particularly accurate simulation of the energy deposition is achieved.

For example, the effective thickness of the imaged object may be used to simulate the attenuation, scattering, and/or transmission of the imaged object.

According to a further aspect of the present embodiments, a computer-implemented method for processing an X-ray image is provided. Therein, a noise-variance-stabilized X-ray image is generated using a computer-implemented method according to the present embodiments. An image processing algorithm is applied to the noise-variance-stabilized X-ray image. As a result, a processed X-ray image is generated.

The image processing algorithm may, for example, include a denoising algorithm. In this case, the noise-variance-stabilization is particularly beneficial, since the same denoising algorithm may be used for different scenarios and, for example, imaging parameters, even though the noise content differs on the level of the X-ray images before the noise-variance-stabilization.

The image processing algorithm may, alternatively or in addition, include an edge enhancement algorithm and/or a contrast enhancement algorithm and/or a medical segmentation algorithm, etc.

According to a number of implementations, a back-transformed X-ray image is computed by applying an inverse of the variance-stabilizing transformation to the processed X-ray image.

According to a number of implementations, applying the image processing algorithm (e.g., the denoising algorithm) includes applying a trained machine learning model (MLM) to the noise-variance-stabilized X-ray image.

In this case, the present embodiments are particularly beneficial, since the MLM otherwise would have to be trained based on training images according to a huge variety of different imaging parameters and corresponding noise contents in order to be applicable universally. The MLM may, for example, be configured as described in the publication of Hariharan et al. mentioned in the Background of the present disclosure.

In general terms, a trained MLM may mimic cognitive functions that humans associate with other human minds. For example, by training based on training data, the MLM may be able to adapt to new circumstances and to detect and extrapolate patterns. Another term for a trained MLM is “trained function.”

In general, parameters of an MLM may be adapted or updated by training. For example, supervised training, semi-supervised training, unsupervised training, reinforcement learning, and/or active learning may be used. Further, representation learning, also denoted as feature learning, may be used. For example, the parameters of the MLM s may be adapted iteratively by a number of (e.g., several) steps of training. For example, within the training of a certain loss function, also denoted as cost function, may be minimized. For example, within the training of an artificial neural network, ANN, the backpropagation algorithm may be used.

For example, an MLM may include an ANN, a support vector machine, a decision tree, and/or a Bayesian network, and/or the MLM may be based on k-means clustering, Q-learning, genetic algorithms, and/or association rules. For example, an ANN may be or include a deep neural network, a convolutional neural network, or a convolutional deep neural network. Further, an ANN may be an adversarial network, a deep adversarial network, and/or a generative adversarial network.

According to a number of implementations, the MLM is an ANN (e.g., a convolutional neural network, CNN, such as a U-Net).

Such MLM s are proven as image-to-image algorithms in the medical context.

According to a number of implementations, a detector model is calibrated to flat-field images acquired with the X-ray detector, and the noise level parameter is computed depending on the detector model.

According to a further aspect of the present embodiments, an X-ray imaging method is provided. Therein, an X-ray image is generated by an X-ray imaging system (e.g., by an X-ray source and an X-ray detector of the X-ray imaging system), for example, controlled by a control system of the X-ray imaging system. A computer-implemented method for processing the X-ray image according to the present embodiments is carried out based on the X-ray image (e.g., by the control system and/or the data processing system), and the processed X-ray image or an image depending on the processed X-ray image (e.g., the back transformed X-ray image) is displayed by a display device (e.g., a display device of the X-ray imaging system).

According to a number of implementations, the X-ray imaging method is carried out as a fluoroscopy method, where a sequence of consecutive X-ray images including the X-ray image is generated by the X-ray imaging system, and each of the consecutive X-ray images is processed by using a computer-implemented method for processing the X-ray image according to the present embodiments. Each processed X-ray image or an image depending on the respective processed X-ray image is, for example, displayed by the display device.

The reduction of the computational effort by the computer-implemented method for generating a noise-variance-stabilized X-ray image according to the present embodiments is particularly beneficial in fluoroscopy methods (e.g., in case the required computations are carried online or in real-time, at least partially).

According to a number of implementations, a Monte Carlo model of the X-ray imaging system is used to simulate the energy deposition.

According to a number of implementations, the simulation of the energy deposition is performed in real-time.

Further implementations of the X-ray imaging method according to the present embodiments follow directly from the various embodiments of the computer-implemented methods according to the present embodiments, and vice versa. For example, individual features and corresponding explanations as well as advantages relating to the various implementations of the computer-implemented methods according to the present embodiments may be transferred analogously to corresponding implementations of the X-ray imaging method according to the present embodiments.

According to a further aspect of the present embodiments, a data processing system that is configured to carry out a computer-implemented method for generating a noise-variance-stabilized X-ray image according to the present embodiments and/or a computer-implemented method for processing an X-ray image according to the present embodiments is provided.

In the present disclosure, the expressions “data processing system” and “at least one data processing device” may be used interchangeably. A data processing device may, for example, be understood as a data processing device that includes processing circuitry. The data processing device may therefore, for example, process data to perform computing operations. This may also include operations to perform indexed accesses to a data structure (e.g., a look-up table, LUT), as well as a data processing process implemented in hardware.

For example, the data processing device may include one or more computers, one or more microcontrollers, and/or one or more integrated circuits (e.g., one or more application-specific integrated circuits, A SIC, one or more field-programmable gate arrays, FPGA, and/or one or more systems on a chip, SoC). The data processing device may also include one or more processors (e.g., one or more microprocessors, one or more central processing units, CPU, one or more graphics processing units, GPU, and/or one or more signal processors, such as one or more digital signal processors, DSP). The data processing device may also include a physical or a virtual cluster of computers or other of said units.

In various embodiments, the data processing device includes one or more hardware and/or software interfaces and/or one or more memory units.

A memory unit may be implemented as a volatile data memory (e.g., a dynamic random access memory, DRAM or a static random access memory, SRAM) or as a non-volatile data memory (e.g., a read-only memory, ROM, a programmable read-only memory, PROM, an erasable programmable read-only memory, EPROM, an electrically erasable programmable read-only memory, EEPROM, a flash memory or flash EEPROM, a ferroelectric random access memory, FRAM, a magnetoresistive random access memory, MRAM, or a phase-change random access memory, PCRAM).

According to a further aspect of the present embodiments, an X-ray imaging system is provided. The X-ray imaging system includes an X-ray source, an X-ray detector, and a control system that is configured to control the X-ray source and the X-ray detector to generate an X-ray image. The X-ray imaging system further includes a data processing system that is adapted to carry out a computer-implemented method for processing an X-ray image according to the present embodiments.

The control system and the data processing system may be separated from each other. In this case, the control system may be understood to be or include a further data processing system according to the definition above. Alternatively, the data processing system of the X-ray imaging system includes the control system. For example, the control system may correspond to one or more data processing apparatuses of the data processing system.

For example, the X-ray imaging system includes a display device, where the control system is configured to control the display device to display the processed X-ray image or an image depending on the processed X-ray image. In the latter case, the control system or the data processing system may be adapted to generate the image depending on the processed X-ray image. The processing may, for example, include applying one of one or more image processing algorithms mentioned above.

According to a number of implementations, the control system is configured to acquire flat-field images by controlling the X-ray source and the X-ray detector. The data processing system is configured to determine the respective noise level parameters of the acquired flat field images and to compare the noise level parameters of the acquired flat field images with the calculated noise level parameters as a quality check of for the X-ray imaging system.

Further implementations of the X-ray imaging system according to the present embodiments follow directly from the various embodiments of the computer-implemented methods and the X-ray imaging method according to the present embodiments, and vice versa. For example, individual features and corresponding explanations as well as advantages relating to the various implementations of the computer-implemented methods and the X-ray imaging method according to the present embodiments may be transferred analogously to corresponding implementations of the X-ray imaging system according to the present embodiments. For example, the X-ray imaging system according to the present embodiments is configured or programmed to carry out the X-ray imaging method according to the present embodiments. For example, the X-ray imaging system according to the present embodiments carries out the X-ray imaging method according to the present embodiments.

According to a further aspect of the present embodiments, a computer program including instructions is provided. When the instructions are executed by a data processing system, the instructions cause the data processing system to carry out a computer-implemented method according to the present embodiments.

The instructions may be provided as program code, for example. The program code may, for example, be provided as binary code or assembler and/or as source code of a programming language (e.g., C) and/or as program script (e.g., Python).

According to a further aspect of the present embodiments, a further computer program including further instructions is provided. When the further instructions are executed by an X-ray imaging system according to the present embodiments (e.g., by the data processing system of the X-ray imaging system and/or the control system of the X-ray imaging system), the further instructions cause the X-ray imaging system to carry out an X-ray imaging method according to the present embodiments.

The further instructions may be provided as program code, for example. The program code may, for example, be provided as binary code or assembler and/or as source code of a programming language (e.g., C) and/or as program script (e.g., Python).

According to a further aspect of the present embodiments, a computer-readable storage medium storing a computer program and/or a further computer program according to the present embodiments is provided.

The computer program, the further computer program, and the computer-readable storage medium are respective computer program products including the instructions and/or the further instructions.

Further features and feature combinations of the present embodiments are obtained from the figures and their description as well as the claims. For example, further implementations of the present embodiments may not necessarily contain all features of one of the claims. Further implementations of the present embodiments may include features or combinations of features that are not recited in the claims.

In the following, the present embodiments will be explained in detail with reference to specific example implementations and respective schematic drawings. In the drawings, same or functionally same elements may be denoted by the same reference signs. The description of same or functionally same elements is not necessarily repeated with respect to different figures.

### BRIEF DESCRIPTION OF THE DRAWINGS

FIG. 1 shows schematically an example implementation of an X-ray imaging system according to the present embodiments;

FIG. 2 shows a schematic flow diagram of an example implementation of a computer-implemented method for generating a noise-variance-stabilized X-ray image according to the present embodiments;

FIG. 3 shows a schematic flow diagram of an example implementation of a computer-implemented method for processing an X-ray image according to the present embodiments;

FIG. 4 shows a schematic flow diagram of applying a physics model in a further example implementation of a computer-implemented method for generating a noise-variance-stabilized X-ray image according to the present embodiments;

FIG. 5 shows schematically an X-ray source and an X-ray detector of a further example implementation of an X-ray imaging system according to the present embodiments, as well as the imaged object and various processes that affect an X-ray beam in the beam path;

FIG. 6 shows a schematic flow diagram of a further example implementation of a computer-implemented method for generating a noise-variance-stabilized X-ray image according to the present embodiments;

FIG. 7 shows schematically a convolutional neural network;

FIG. 8 shows schematically an artificial neural network; and

FIG. 9 shows schematically a further convolutional neural network.

### DETAILED DESCRIPTION

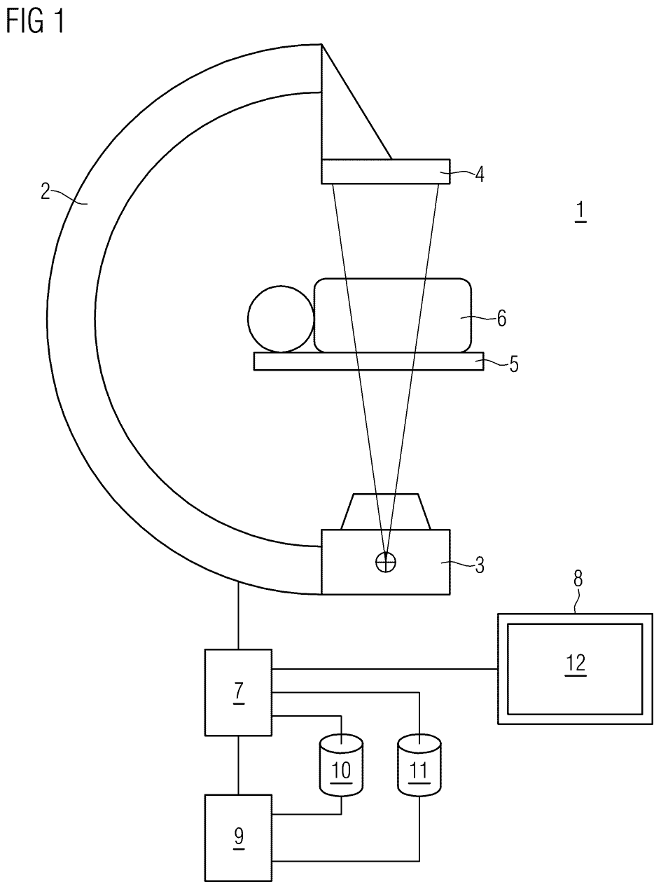

FIG. 1 shows schematically an example implementation of an X-ray imaging system 1 according to the present embodiments. The X-ray imaging system 1 includes a source unit 3 with an X-ray source, a detector unit 4 with an X-ray detector, and a control system 7 that is configured to control the X-ray source and the X-ray detector to generate X-ray images 13 depicting an object 6 (e.g., a patient).

The X-ray imaging system 1 may, for example, include a patient table 5, on which the object 6 is arranged. The X-ray imaging system 1 includes a data processing system 9 according to the present embodiments that is adapted to carry out a computer-implemented method for processing the X-ray image 13 according to the present embodiments. In the following, a number of (e.g., several) functions and method acts are described to be carried out by the control system 7, while other functions and method acts are described to be carried out by the data processing system 9. It is noted that the functions and method acts may also be distributed in different ways in alternative implementations.

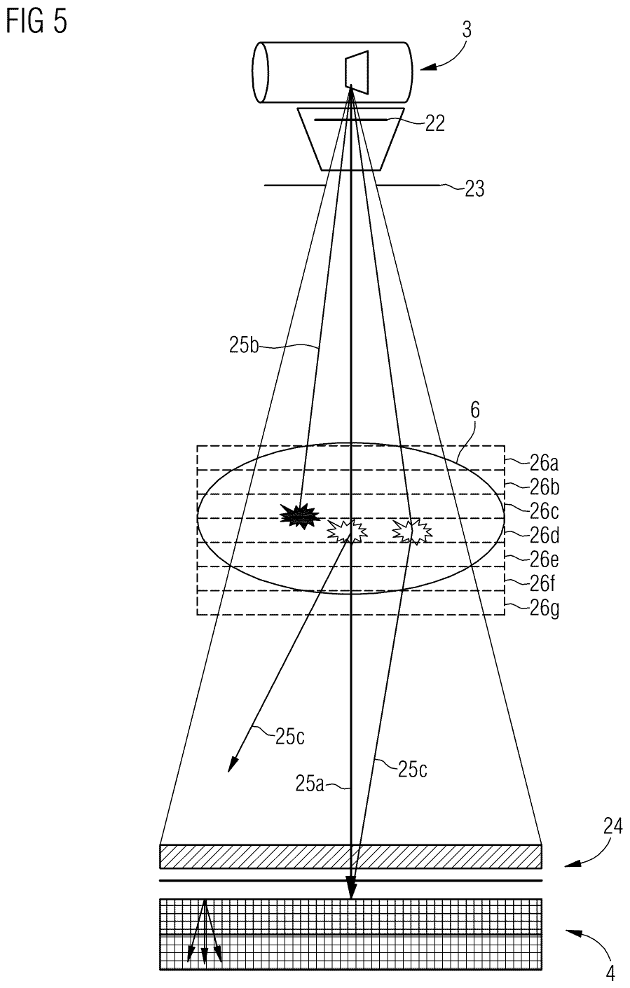

For example, the control system 7 may adjust various imaging parameters of the X-ray imaging system 1 including, for example, exposure parameters such as a peak kilovoltage of the X-ray source, a tube current of the X-ray source, and/or an X-ray pulse duration. For example, the control system 7 may adjust further imaging parameters such as a filter material and/or filter thickness of an X-ray filter 22 (e.g., a copper filter) by placing the appropriate X-ray filter 22 into the beam path or by removing the appropriate X-ray filter 22 from the beam path, respectively (see, e.g., FIG. 5). For example, the control system 7 may adjust further imaging parameters such as a collimator opening size of an X-ray collimator 23. For example, the control system 7 may bring the X-ray collimator 23 into the beam path or remove the X-ray collimator 23 from the beam path, respectively (see, e.g., FIG. 5). For example, the control system 7 may adjust further imaging parameters such as a gain factor of the X-ray detector. For example, the control system 7 may bring an anti-scattering grid 24 into the beam path or may remove the anti-scattering grid 24 from the beam path, respectively (see, e.g., FIG. 5). For example, the control system 7 may adjust a gain factor of the X-ray detector.

In some implementations, the X-ray imaging system 1 includes a display device 8, where the control system 7 is configured to control the display device 8 to display the processed X-ray image 12 or an image depending on the processed X-ray image 12. In case a fluoroscopy-based medical intervention is carried out, the X-ray images 13 are, for example, generated as a sequence of consecutive frames to allow an operator to monitor or supervise the medical intervention. The X-ray imaging system 1 is, for example, configured to carry out an X-ray imaging method according to the present embodiments, where an X-ray image 13 is generated by the X-ray imaging system 1 using the X-ray source and the X-ray detector, and a computer-implemented method for processing the X-ray image 13 is carried out (e.g., by the data processing system 9). The processed X-ray image 12 or the image depending on the processed X-ray image 12 is displayed on the display device 8.

In some implementations, the X-ray imaging system 1 is, as indicated in FIG. 1, configured as a fluoroscopy system (e.g., a C-arm fluoroscopy system), where the source unit 3 and the detector unit 4 are attached opposite to each other on a C-arm 2 that may be rotated around different axes. The corresponding motions are denoted as angular and orbital motion, respectively. In some implementations, apart from the rotational motion of the C-arm 2, the patient table 5 and the C-arm 2 may be positioned relative to each other by respective translatory motions of the C-arm 2 and/or the patient table 5. Consequently, the position and/or orientation of the X-ray source with respect to the object 6 and the position and/or orientation of the X-ray detector with respect to the object 6 may be adjusted exactly to the required imaging perspective.

In some implementations, the X-ray imaging system 1 (e.g., the data processing system 9) may includes databases 10, 11 to store and/or read data in the course of the computer-implemented method according to the present embodiments and/or for other purposes.

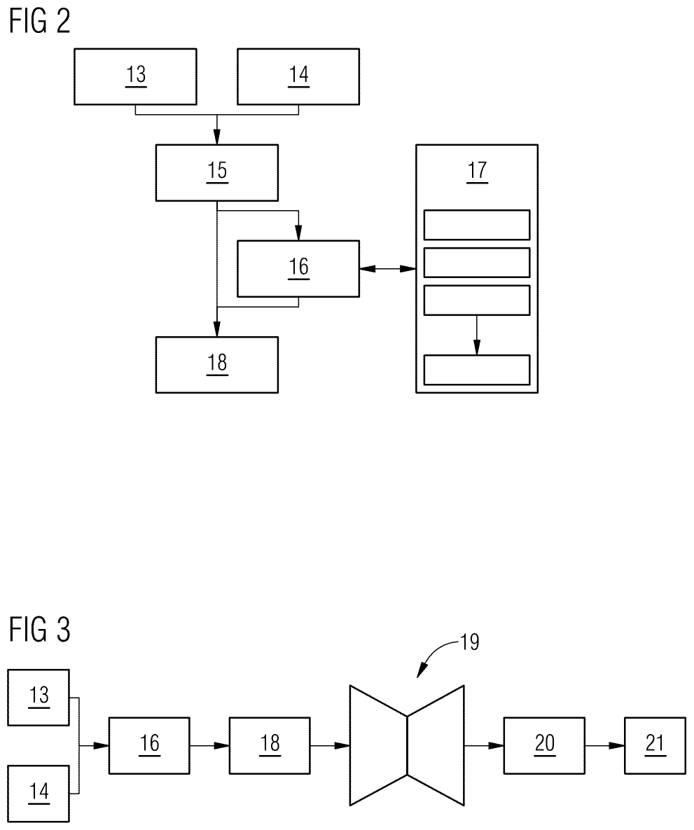

FIG. 2 shows a schematic flow diagram of an example implementation of a computer-implemented method for generating a noise-variance-stabilized X-ray image according to the present embodiments.

T herein, the X-ray image 13 and a corresponding set of imaging parameters 14 of the X-ray imaging system 1 are received (e.g., by the data processing system 9) and, for example, fed to the imaging, processing pipeline 15. The imaging parameters 14 may include the peak kilovoltage of the X-ray source, the tube current of the X-ray source, and/or the X-ray pulse duration. A noise level parameter 16 (e.g., the value α=(σ²−σₑ²)/yₘ) is computed by using a physics model 17 to, for example, simulate an energy deposition of X-ray quanta emitted by the X-ray source on the X-ray detector depending on the set of imaging parameters 14. A noise-variance-stabilized X-ray image 18 is generated by applying a variance-stabilizing transformation (e.g., a general Anscombe transform (GAT)) that depends on the noise level parameter, to the X-ray image 13. The GAT is, for example, computed according to

$${y}^{′} = \frac{2}{α} \sqrt{α \cdot y + \frac{3}{8} {α}^{2} + {σ}_{n}^{2}} ,$$

where y denotes a pixel value of the X-ray image 13, y′ denotes the respective pixel value of the noise-variance-stabilized X-ray image 18, and σₙ² denotes the variance of the electronic noise of the X-ray detector, which is predetermined, for example, during a calibration phase.

The noise level function in two-dimensional X-ray imaging is, for example, given by

$${σ}^{2} = α \cdot {y}_{m} + {{σ}_{e}}^{2} ,$$

the first term denoting the variance of the quantum noise and the second term denoting the variance of the electronic noise. The variance of the quantum noise may, in principle, take any value, depends on the gray value yₘ (e.g., not corrupted by noise), and depends, via α, on the Imaging parameters 14 (e.g., the spectral distribution of X-ray quanta reaching the X-ray detector surface and also on the X-ray detector's properties). It is therefore relatively hard to estimate. Consequently, the noise level parameter α is determined using the physics model 17, as described. The electronic noise essentially depends on the X-ray detector's properties only and may be determined by measurements for every individual detector mode.

The imaging parameters 14 may, for example, include the peak kilovoltage, the tube current, the X-ray pulse duration, the filter material and/or the filter thickness, the collimator opening size, the gain factor, and/or the presence or absence of the anti-scattering grid 24 in the beam path. The imaging parameters 14 may include the respective position and/or angulation (e.g., in terms of the angular angle and the orbital angle of the C-arm 2) of the X-ray source and the X-ray detector with respect to the object 6, and/or the position and/or orientation of the patient table 5. The imaging parameters 14 may include an effective thickness of the imaged object 6 (e.g., in terms of its water-equivalent thickness (WET)).

FIG. 3 shows a schematic flow diagram of an example implementation of a computer-implemented method for processing the X-ray image 13 according to the present embodiments. Therein, the noise-variance-stabilized X-ray image 18 is generated, as described, for example, with respect to FIG. 2.

Then, an image processing algorithm (e.g., an MLM 19 trained for denoising X-ray images) is applied to the noise-variance-stabilized X-ray image 18, and a back-transformed X-ray image 21 is generated by applying the inverse variance-stabilizing transformation to the output image 20 resulting as an output of the image processing algorithm. The back-transformed X-ray image 21 may also be denoted as processed X-ray image 21 and may be understood as a result of the method for processing the X-ray image 13.

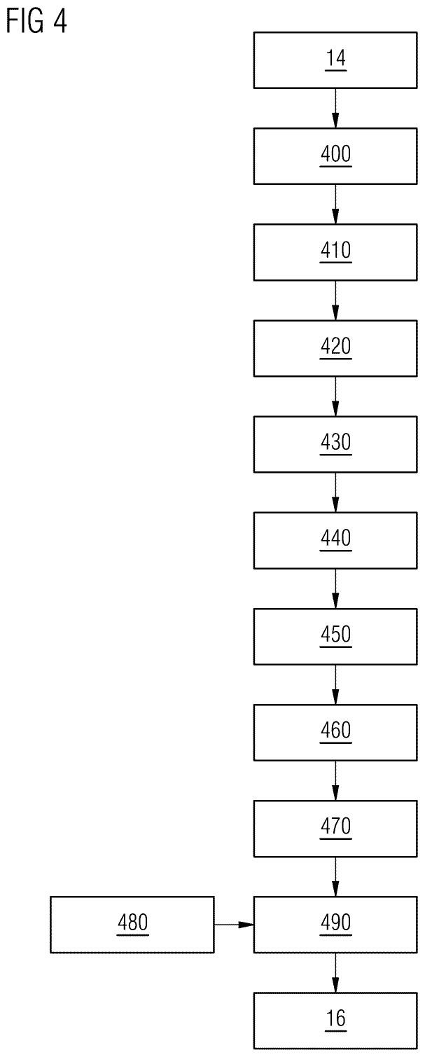

In the following, it is explained in more detail how the physics model 17 is applied to compute the noise level parameter. FIG. 4 shows a corresponding schematic flow diagram, and FIG. 5 shows schematically some simulated processes.

In act 400, the emission of X-ray quanta from the X-ray source (e.g., X-ray tube) is simulated or estimated (e.g., using the computational toolkit spektr 3.0). The result is an initial energy spectrum. If the X-ray filter 22 is in the beam path, the change of the energy spectrum by the X-ray filter 22 is computed in act 410. Acts 400 and 410 may also be combined. Act 410 may also be carried out using spektr 3.0. In act 420, the effect of the collimator 23 on the resulting energy spectrum is simulated (e.g., using a Monte Carlo simulation).

In act 430, the effect of an interaction (e.g., scattering, absorption, and transmission) of X-ray quanta with an additional object located between the X-ray source and the imaged object 6 (e.g., between the collimator 23 and the image object 6) on the resulting energy spectrum is simulated, in case such additional object is present. Also, a Monte Carlo simulation may be used. Depending on the position of the C-arm 2, the patient table 5 may, for example, be located between the X-ray source and the imaged object 6. In act 440, the effect of an interaction (e.g., scattering, absorption, and transmission) of X-ray quanta with the imaged object 6 itself on the resulting energy spectrum is simulated (e.g., also by using one or more Monte Carlo simulation steps). In the example of FIG. 5, the image object 6 is virtually divided into a number of (e.g., several) slices 26a, 26b, 26c, 26d, 26e, 26f, 26g and a separate Monte Carlo simulation step is used to simulate the interaction in each slice 26a, 26b, 26c, 26d, 26e, 26f, 26g. For visualization purposes, X-ray quanta 25a that are transmitted through the imaged object 6, X-ray quanta 25c that are scattered in the imaged object 6, as well as X-ray quanta 25b that are absorbed in the imaged object 6 are shown schematically.

In act 450, the effect of an interaction (e.g., scattering, absorption, and transmission) of X-ray quanta with an additional object located between the imaged object 6 and the X-ray detector 4 (e.g., between the image object 6 and the anti-scattering grid 24) on the resulting energy spectrum is simulated in case such additional object is present. Also, a Monte Carlo simulation may be used. Depending on the position of the C-arm 2, the patient table 5 may, for example, be located between the imaged object 6 and the X-ray detector. In act 460, the effect of an interaction (e.g., scattering, absorption, and transmission) of X-ray quanta with the anti-scattering grid 24 on the resulting energy spectrum is simulated. Also, a Monte Carlo simulation may be used. In act 490, the energy deposition on the surface of the X-ray detector (e.g., the E2E-value) is determined based on the finally resulting energy spectrum. Also, a Monte Carlo simulation may be used.

In act 490, the noise-variance parameter α is computed based on the energy deposition (e.g., based on the E2E-value by using calibration measurements (act 480) and adapting them according to a suitable X-ray detector model; for example, are cascaded linear systems models as the models of Cunningham at al. or Punnoose et al. mentioned in the Background). For example, the noise level parameter may be computed according to the equation

$$α = b \cdot ( {c}_{0} + {c}_{1} \frac{\overline{{E}^{2}}}{\overline{E}} ) ,$$

where b, c₀, and c₁ are parameters obtained from the calibration measurements of act 480, and the E2E-value is, for example, computed as

$$\frac{\overline{{E}^{2}}}{\overline{E}} = \frac{\int ( 1 - exp ( - {μ}_{c , pe} ( E ) {t}_{c} ) ) {E}^{2} {q}_{0} ( E ) dE}{\int ( 1 - exp ( - {μ}_{c , pe} ( E ) {t}_{c} ) ) {Eq}_{0} ( E ) dE} ,$$

with μ_{c,pe} denoting the attenuation coefficient due to the photo-effect of the X-ray converter (e.g., scintillator) of the X-ray detector, and q₀(E) is the number of X-ray quanta per square-area per energy bin incident to the X-ray converter obtained from the last Monte Carlo simulation step. Using the model in act 490 effectively includes a conversion of X-ray quanta to optical photons, a conversion of the optical photons to detector signals, and a conversion of the detector signals to respective pixel signal information.

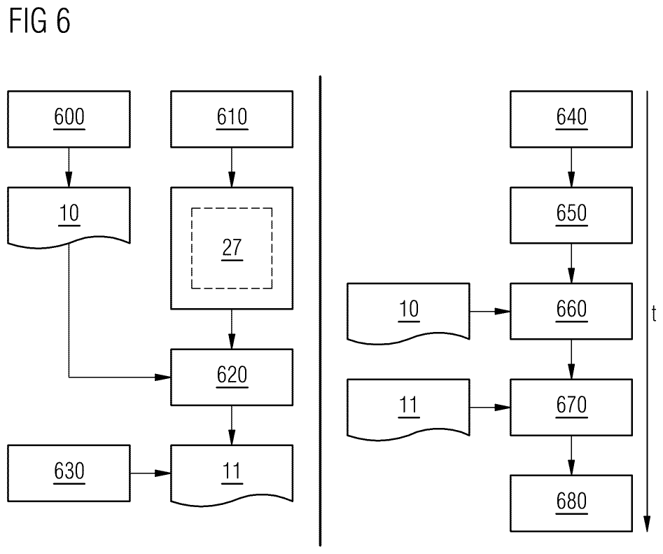

FIG. 6 shows a schematic flow diagram of a further example implementation of a computer-implemented method for generating a noise-variance-stabilized X-ray image according to the present embodiments. In the shown implementation, the simulations and modelling as described, for example, with respect to FIG. 5 are performed offline, and the results are stored in the databases 10, 11 and implemented on the X-ray imaging system 1 via lookup tables and/or fit functions. For example, it is also possible to interpolate the data provided in the lookup tables according to the current requirements and system. Due to computational efforts and run time, especially of the Monte Carlo simulations, such implementations may be beneficial. However, in alternative implementations, the simulations and modeling are performed online in real-time, and the results are transferred to the imaging pipeline in real-time.

For example, acts 600 to 630 of FIG. 6 may be carried out by the data processing system 9, while the acts 640 to 680 may be carried out by the control system 7. However, this distribution may differ in other implementations. Act 600 of FIG. 6 corresponds to acts 400 to 470 of FIG. 4, resulting in the E2E-value, which is stored to the database 10. Act 610 of FIG. 6 corresponds to act 480 of FIG. 4, resulting in the respective calibration measurement data 27. These are, for example, obtained by an analysis of multiple flat field images without imaging an object for many different values and combinations of the imaging parameters 14.

In act 620, the parameters b, c₀, and c₁ are obtained by fitting the calibration measurement data 27 to the equation for a above. In optional act 630, the influence of different image conditioning algorithms on the parameters b, c₀, and c₁ may be simulated. As a result, the various values for the parameters b, c₀, and c₁ may be stored to the database 11.

Acts 640 to 680 may, for example, be carried out in real-time during the fluoroscopy. In act 640, the imaging parameters 14 concerning the X-ray source, the X-ray filter 22, the collimator 23, and the X-ray detector are read for the current frame (e.g., the peak kilovoltage the tube current, the X-ray pulse duration, the filter material and/or the filter thickness, the collimator opening size, the gain factor, and/or the presence or absence of the anti-scattering grid 24 in the beam path). In act 640, the imaging parameters 14 concerning the geometric arrangement and the object 6 (e.g., position and/or angulation of the C-arm 2, the position and/or orientation of the patient table 5, and the WET) are read for the current frame. In act 660, the corresponding E2E-value is read from the database 10 for the current set of imaging parameters 14, and in act 670, the corresponding parameters b, c₀, and c₁ are read from the database 11 for the current set of imaging parameters 14. In act 680, the noise level parameter α is computed for the current frame depending on the read-out E2E-value, and the read-out parameters b, c₀, and c₁ according to the equation above.

As described, according to the present embodiments, a noise-variance-stabilized X-ray image 18 is obtained in a way that reduces the draw backs of data-driven approaches.

In a number of (e.g., several) implementations, the described method uses a physics model 17 that may be integrated into the X-ray imaging system 1, to estimate the NLF of the image chain (e.g., the noise level parameter α). This leads to various advantages. For example, the determination of the NLF may be performed very fast and accurately and independent of the X-ray image 13 itself. The method is robust even in complex imaging scenarios where data-driven approaches may fail to determine the NLF reliably. The computational efforts, and therefore computational hardware requirements, may be relaxed, leading to potential cost savings. This is also suitable for cost-efficient mobile C-arm systems.

In some embodiments, an implementation of the model-driven approach in parallel to a data-driven approach may provide a back-up or a possibility for consistency checks, thus reducing the occurrence of image artefacts due to non-optimal parameterization of image processing algorithms. A n interchangeable implementation of data-driven and the model-based approach would allow to select either one of the two methods depending on the actual imaging situation.

Since the noise level parameter α is sensitive to spectral changes of the X-ray source's output and X-ray detector's efficiency, the proposed method may, in some implementations, also be used to monitor and track the status of the X-ray imaging system 1, detect potential abnormal states, defects, or degradation or aging of components, and may also be used for system calibration purposes.

As the calculation of the Noise Level Function may be done independent or prior to acquiring the X-ray image 13, the method may, in some implementations, also be used to predict the NLF even before real images are acquired. This may help to improve X-ray exposure control methods.

In some implementations, errors in the output of sensors (e.g., the status of anti-scattering grid 23) and exposure parameters may also be predicted. This is possible by analyzing system parameters such as the peak kilovoltage, the filter thickness, the WET, the status of the anti-scattering grid 23, and the estimated a. In addition, a data-driven estimate may be used for improving the prediction of status of anti-scattering grid 23 and/or the WET, in some implementations. The status of anti-scattering grid 23 may help for performing a scatter correction later in the image processing pipeline, for example.

In some implementations, the evaluations may, for example, be done regularly as part of a calibration of the X-ray detector 4. Flat images (e.g., without ab object to be imaged) may be acquired at specified exposure parameters (e.g., combinations of peak kilovoltage, rube current, and spectral filter). The values for a may be computed from variance and mean of the images. The value may be compared with the value estimated from the look up table. If there are deviations in the value, this may indicate a failure or malfunction of the X-ray source 3, the X-ray detector 4, the anti-scattering grid, and so forth. By acquiring a number of (e.g., several) flat images at different spectra, it may, for example, be identified (e.g., along with additional information from the achieved exposure parameters and WET estimation) whether the X-ray spectrum at the X-ray source 3 has changed or the response of the X-ray detector 4 has changed. Alternatively or in addition, damage of the anti-scattering grid may be predicted.

The MLM 19 depicted in FIG. 3 may, for example, be an ANN.

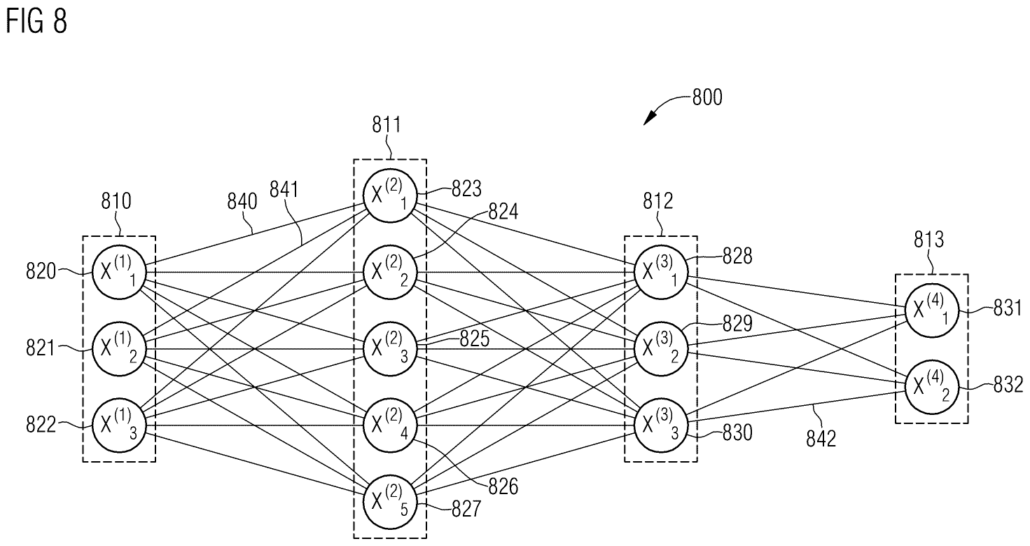

FIG. 8 displays an embodiment of an ANN 800 that is, for example, configured as an MLP. The ANN 800 includes nodes 820, . . . , 832 and edges 840, . . . , 842, where each edge 840, . . . , 842 is a directed connection from a first node 820, . . . , 832 to a second node 820, . . . , 832. In general, the first node 820, . . . , 832 and the second node 820, . . . , 832 are different nodes 820, . . . , 832. It is, however, also possible that the first node 820, . . . , 832 and the second node 820, . . . , 832 are the same. For example, in FIG. 8, the edge 840 is a directed connection from the node 820 to the node 823, and the edge 842 is a directed connection from the node 830 to the node 832. An edge 840, . . . , 842 from a first node 820, . . . , 832 to a second node 820, . . . , 832 is also denoted as ingoing edge for the second node 820, . . . , 832 and as outgoing edge for the first node 820, . . . , 832.

In this example, the nodes 820, . . . , 832 of the artificial neural network 800 may be arranged in layers 810, . . . , 813, where the layers may include an intrinsic order introduced by the edges 840, . . . , 842 between the nodes 820, . . . , 832. For example, edges 840, . . . , 842 may exist only between neighboring layers of nodes. In the displayed example, there is an input layer 810 including only nodes 820, . . . , 822 without an incoming edge, an output layer 813 including only nodes 831, 832 without outgoing edges, and hidden layers 811, 812 in-between the input layer 810 and the output layer 813. In general, the number of hidden layers 811, 812 may be chosen arbitrarily. In an MLP, this number is at least one. The number of nodes 820, . . . , 822 within the input layer 810 may relate to the number of input values of the artificial neural network 800, and the number of nodes 831, 832 within the output layer 813 may relate to the number of output values of the artificial neural network 800.

For example, a real number may be assigned as a value to every node 820, . . . , 832 of the artificial neural network 800. Here, x⁽ⁿ⁾ᵢ denotes the value of the i-th node 820, . . . , 832 of the n-th layer 810, . . . , 813. The values of the nodes 820, . . . , 822 of the input layer 810 are equivalent to the input values of the artificial neural network 800. The values of the nodes 831, 832 of the output layer 813 are equivalent to the output value of the artificial neural network 800. Further, each edge 840, . . . , 842 may include a weight being a real number. For example, the weight is a real number within the interval [−1, 1] or within the interval [0, 1]. Here, w^{(m,n)}_{i,j} denotes the weight of the edge between the i-th node 820, . . . , 832 of the m-th layer 810, . . . , 813 and the j-th node 820, . . . , 832 of the n-th layer 810, . . . , 813. Further, the abbreviation w⁽ⁿ⁾_{i,j} is defined for the weight w^{(n,n+1)}_{i,j}. For example, to calculate the output values of the neural network 800, the input values are propagated through the neural network 800. For example, the values of the nodes 820, . . . , 832 of the (n+1)-th layer 810, . . . , 813 may be calculated based on the values of the nodes 820, . . . , 832 of the n-th layer 810, . . . , 813 by

$${x}_{j}^{( n + 1 )} = f ( {\sum }_{i} {x}_{i}^{( n )} {w}_{i , j}^{( n )} ) .$$

Herein, the function f is denoted as transfer function or activation function. Known transfer functions are step functions, the sigmoid functions (e.g., the logistic function), the generalized logistic function, the hyperbolic tangent, the arctangent function, the error function, the smoothstep function, or rectifier functions. The transfer function is, for example, used for normalization purposes. For example, the values are propagated layer-wise through the neural network 800, where values of the input layer 810 are given by the input of the neural network 800, where values of the first hidden layer 811 may be calculated based on the values of the input layer 810 of the neural network 800, where values of the second hidden layer 812 may be calculated based on the values of the first hidden layer 811, and so forth.

In order to set the values w^{(m,n)}_{i,j} for the edges, the neural network 800 is to be trained using training data. For example, training data includes training input data and training output data (e.g., denoted as tᵢ). For a training step, the neural network 800 is applied to the training input data to generate calculated output data. For example, the training data and the calculated output data include a number of values. The number of values is equal with the number of nodes of the output layer. For example, a comparison between the calculated output data and the training data is used to recursively adapt the weights within the neural network 800 (e.g., backpropagation algorithm). For example, the weights are changed according to

$${w}_{i , j}^{′ ( n )} = {w}_{i , j}^{( n )} - {γδ}_{j}^{( n )} {x}_{i}^{( n )} ,$$

where γ is a predefined learning rate, and the numbers δ⁽ⁿ⁾ⱼ may be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

based on δ⁽ⁿ⁺¹⁾ⱼ, if the (n+1)-th layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)-th layer is the output layer 813, where f′ is the first derivative of the activation function, and t⁽ⁿ⁺¹⁾ⱼ is the comparison training value for the j-th node of the output layer 813.

The ML M 19 may, for example, be a CNN. A convolutional neural network, CNN, is an ANN that uses a convolution operation instead of general matrix multiplication in at least one of its layers. These layers are denoted as convolutional layers. For example, a convolutional layer performs a dot product of one or more convolution kernels with the convolutional layer's input data, where the entries of the one or more convolution kernel are parameters or weights that may be adapted by training. For example, one may use the Frobenius inner product and the ReLU activation function. A convolutional neural network may include additional layers (e.g., pooling layers, fully connected layers, and/or normalization layers).

By using convolutional neural networks, the input may be processed in a very efficient way because a convolution operation based on different kernels may extract various image features so that by adapting the weights of the convolution kernel the relevant image features may be found during training. Further, based on the weight-sharing in the convolutional kernels, fewer parameters are to be trained, which prevents overfitting in the training phase and allows to have faster training or more layers in the network, improving the performance of the network.

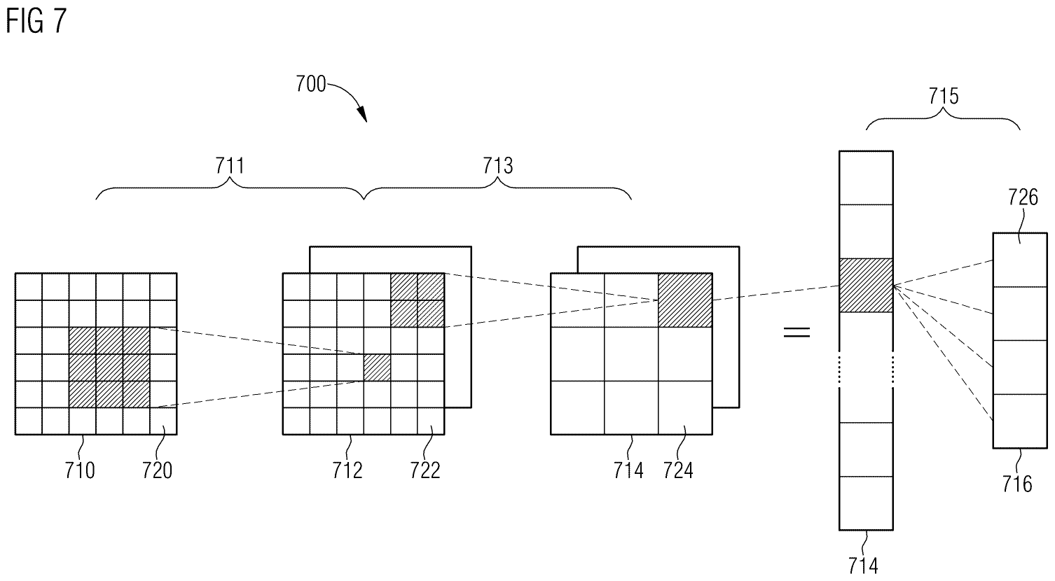

FIG. 7 displays an example embodiment of a convolutional neural network 700. In the displayed embodiment, the convolutional neural network 700 includes an input node layer 710, a convolutional layer 711, a pooling layer 713, a fully connected layer 714, and an output node layer 716, as well as hidden node layers 712, 714. Alternatively, the convolutional neural network 200 may include a number of (e.g., several) convolutional layers 711, a number of (e.g., several) pooling layers 713, and/or a number of (e.g., several) fully connected layers 715, as well as other types of layers. The order of the layers may be chosen arbitrarily; fully connected layers 715 may be used as the last layers before the output layer 716.

For example, within a convolutional neural network 700, nodes 720, 722, 724 of a node layer 710, 712, 714 may be considered to be arranged as a d-dimensional matrix or as a d-dimensional image. For example, in the two-dimensional case, the value of the node 720, 722, 724 indexed with i and j in the n-th node layer 710, 712, 714 may be denoted as x(n)[i, j]. However, the arrangement of the nodes 720, 722, 724 of one node layer 710, 712, 714 does not have an effect on the calculations executed within the convolutional neural network 700 as such, since these are given solely by the structure and the weights of the edges.

A convolutional layer 711 is a connection layer between an anterior node layer 710 with node values x(n−1) and a posterior node layer 712 with node values x(n). For example, a convolutional layer 711 is characterized by the structure and the weights of the incoming edges forming a convolution operation based on a certain number of kernels. For example, the structure and the weights of the edges of the convolutional layer 711 are chosen such that the values x(n) of the nodes 722 of the posterior node layer 712 are calculated as a convolution x(n)=K*x(n−1) based on the values x(n−1) of the nodes 720 anterior node layer 710, where the convolution * is defined in the two-dimensional case as

$${x}^{( n )} [ i , j ] = ( K * {x}^{( n - 1 )} ) [ i , j ] = {\sum }_{i ′} {\sum }_{j ′} K [ {i}^{′} , {j}^{′} ] \cdot {x}^{( n - 1 )} [ i - {i}^{′} , j - {j}^{′} ] .$$

Herein, the kernel K is a d-dimensional matrix (e.g., in the present example, a two-dimensional matrix) that may be small compared to the number of nodes 720, 722 (e.g., a 3×3 matrix or a 5×5 matrix). For example, this implies that the weights of the edges in the convolution layer 711 are not independent, but chosen such that the weights produce the convolution equation. For example, for a kernel being a 3×3 matrix, there are only 9 independent weights, each entry of the kernel matrix corresponding to one independent weight, regardless of the number of nodes 720, 722 in the anterior node layer 710 and the posterior node layer 712.

In general, convolutional neural networks 700 use node layers 710, 712, 714 with a plurality of channels, for example, due to the use of a plurality of kernels in convolutional layers 711. In those cases, the node layers may be considered as (d+1)-dimensional matrices, the first dimension indexing the channels. The action of a convolutional layer 711 is then in a two-dimensional example defined as

$${x}_{b}^{( n )} [ i , j ] = {\sum }_{a} ( {K}_{a , b} * {x}_{a}^{( n - 1 )} [ i , j ] = {\sum }_{a} {\sum }_{i ′} {\sum }_{j ′} {K}_{a , b} [ {i}^{′} , {j}^{′} ] \cdot {x}_{a}^{( n - 1 )} [ i - {i}^{′} , j - {j}^{′} ] ,$$

where xₐ⁽ⁿ⁾ corresponds to the a-th channel of the anterior node layer 710, x_{b}⁽ⁿ⁾ corresponds to the b-th channel of the posterior node layer 712, and K_{a,b} corresponds to one of the kernels. If a convolutional layer 711 acts on an anterior node layer 710 with A channels and outputs a posterior node layer 712 with B channels, there are A·B independent d-dimensional kernels K_{a,b}.

In general, in convolutional neural networks 700, activation functions may be used. In this embodiment, rectified linear unit (ReLU) is used, with R(z)=max(0, z), so that the action of the convolutional layer 711 in the two-dimensional example is

$${x}_{b}^{( n )} [ i , j ] = R ( {\sum }_{a} ( {K}_{a , b} * {x}_{a}^{( n - 1 )} [ i , j ] ) = R ( {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ) .$$

It is also possible to use other activation functions (e.g., exponential linear unit (ELU)), LeakyReLU, Sigmoid, Tanh, or Softmax.

In the displayed embodiment, the input layer 710 includes 36 nodes 720, arranged as a two-dimensional 6×6 matrix. The first hidden node layer 712 includes 72 nodes 722, arranged as two two-dimensional 6×6 matrices, each of the two matrices being the result of a convolution of the values of the input layer with a 3×3 kernel within the convolutional layer 711. Equivalently, the nodes 722 of the first hidden node layer 712 may be interpreted as arranged as a three-dimensional 2×6×6 matrix, where the first dimension correspond to the channel dimension.

An advantage of using convolutional layers 711 is that spatially local correlation of the input data may be exploited by enforcing a local connectivity pattern between nodes of adjacent layers (e.g., by each node being connected to only a small region of the nodes of the preceding layer).

A pooling layer 713 is a connection layer between an anterior node layer 712 with node values x(n−1) and a posterior node layer 714 with node values x(n). For example, a pooling layer 713 may be characterized by the structure and the weights of the edges and the activation function forming a pooling operation based on a non-linear pooling function f. For example, in the two-dimensional case, the values x(n) of the nodes 724 of the posterior node layer 714 may be calculated based on the values x(n−1) of the nodes 722 of the anterior node layer 712 as

$${x}_{b}^{( n )} [ i , j ] = f ( {x}_{b}^{( n - 1 )} [ {id}_{1} , {jd}_{2} ] , ... , {x}_{b}^{( n - 1 )} [ ( i + 1 ) {d}_{1} - 1 , ( j + 1 ) {d}_{2} - 1 ] ) .$$

In other words, by using a pooling layer 713, the number of nodes 722, 724 may be reduced by replacing a number d1·d2 of neighboring nodes 722 in the anterior node layer 712 with a single node 722 in the posterior node layer 714 being calculated as a function of the values of the number of neighboring nodes. For example, the pooling function f may be the max-function, the average or the L2-Norm. For example, for a pooling layer 713, the weights of the incoming edges are fixed and are not modified by training.

The advantage of using a pooling layer 713 is that the number of nodes 722, 724 and the number of parameters is reduced. This leads to the amount of computation in the network being reduced and to a control of overfitting.

In the displayed embodiment, the pooling layer 713 is a max-pooling layer, replacing four neighboring nodes with only one node, the value being the maximum of the values of the four neighboring nodes. The max-pooling is applied to each d-dimensional matrix of the previous layer. In this embodiment, the max-pooling is applied to each of the two two-dimensional matrices, reducing the number of nodes from 72 to 18.

In general, the last layers of a convolutional neural network 700 may be fully connected layers 715. A fully connected layer 715 is a connection layer between an anterior node layer 714 and a posterior node layer 716. A fully connected layer 713 may be characterized by the fact that a majority of (e.g., all) edges between nodes 714 of the anterior node layer 714 and the nodes 716 of the posterior node layer are present, and the weight of each of these edges may be adjusted individually.

In this embodiment, the nodes 724 of the anterior node layer 714 of the fully connected layer 715 are displayed both as two-dimensional matrices, and additionally as non-related nodes, indicated as a line of nodes, where the number of nodes was reduced for a better presentability. This operation is also denoted as flattening. In this embodiment, the number of nodes 726 in the posterior node layer 716 of the fully connected layer 715 is smaller than the number of nodes 724 in the anterior node layer 714. Alternatively, the number of nodes 726 may be equal or larger.

Further, in this embodiment, the Softmax activation function is used within the fully connected layer 715. By applying the Softmax function, the sum the values of all nodes 726 of the output layer 716 is 1, and all values of all nodes 726 of the output layer 716 are real numbers between 0 and 1. For example, if using the convolutional neural network 700 for categorizing input data, the values of the output layer 716 may be interpreted as the probability of the input data falling into one of the different categories.

For example, convolutional neural networks 700 may be trained based on the backpropagation algorithm. For preventing overfitting, methods of regularization may be used (e.g., dropout of nodes 720, . . . , 724, stochastic pooling, use of artificial data, weight decay based on the L1 or the L2 norm, or max norm constraints).

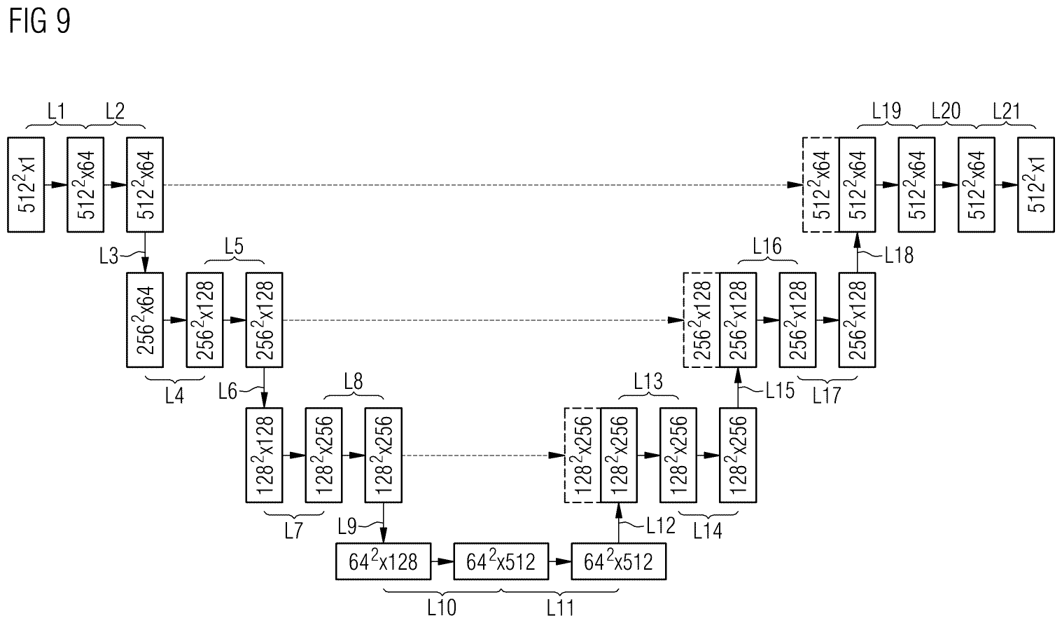

In the example of FIG. 9, the MLM is a CNN (e.g., a convolutional neural network having a U-Net structure). In the displayed example, the input data to the CNN is a two-dimensional medical image including 512×512 pixels, every pixel including one intensity value. The CNN includes convolutional layers indicated by solid, horizontal arrows, pooling layers indicating by solid arrows pointing down, and upsampling layers indicated by solid arrows pointing up. The number of the respective nodes is indicated within the boxes. Within the U-Net structure, first, the input images are downsampled (e.g., by decreasing the size of the images and increasing the number of channels). Afterwards, the input images are upsampled (e.g., by increasing the size of the images and decreasing the number of channels) to generate a transformed image.

All except the last convolutional layers L1, L2, L4, L5, L.7, L8, L10, L11, L13, L14, L16, L17, L19, L20 use 3×3 kernels with a padding of 1, the ReLU activation function, and a number of filters or convolutional kernels that matches the number of channels of the respective node layers as indicated in FIG. 9. The last convolutional layer uses a 1×1 kernel with no padding and the ReLU activation function.

The pooling layers L3, L6, L9 are max-pooling layers, replacing four neighboring nodes with only one node. The value is the maximum of the values of the four neighboring nodes. The upsampling layers L12, L15, L18 are transposed convolution layers with 3×3 kernels and stride 2, which effectively quadruple the number of nodes. The dashed horizontal arrows correspond to concatenation operations, where the output of a convolutional layer L2, L5, L8 of the downsampling branch of the U-Net structure is used as additional inputs for a convolutional layer L13, L16, L19 of the upsampling branch of the U-Net structure. This additional input data is treated as additional channels in the input node layer for the convolutional layer L13, L16, L19 of the upsampling branch.

For training the CNN, a database of 500 first medical images may, for example, be used, where the respective segmentation mask may be created based on annotations of expert radiologists. For example, for each of the 500 first medical images, a segmentation mask may be determined for a structure of interest, where a value of 1 may be assigned to pixels corresponding to the structure of interest, and a value of 0 may be assigned to pixels not corresponding to the structure of interest. The database may be split into training data (e.g., 320 datasets), validation data (e.g., 80 datasets), and test data (e.g., 100 datasets). For training the CNN, the backpropagation algorithm may be used based on a binary cross-entropy cost function

$$L ( x , y ) = {\sum }_{i} {\sum }_{j} BCE ( y [ i , j ] , M ( x ) [ i , j ] )$$

$$with$$

$$BCE ( a , b ) := - a log ( b ) ( b ) - ( 1 - a ) log ( 1 - b ) ,$$

where x denotes a first medical image, y determines the corresponding segmentation mask created by the expert radiologist, and M(x) denotes the result of applying the CNN to the first input medical image x. Alternatively, other cost functions, such as weighted binary cross entropy, Focal Loss, or Dice Loss, may be used.

Based on the validation set of 80 datasets and the corresponding annotations, the best performing machine learning model out of the number of (e.g., several) machine learning models (e.g., with different hyperparameters, such as number of layers, size and number of kernels, padding) may be selected. The specificity and the sensitivity may be determined based on the test set including 100 datasets and the corresponding annotations.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for generating a noise-variance-stabilized X-ray image, the computer-implemented method comprising:
receiving an X-ray image generated by an X-ray imaging system and a corresponding set of imaging parameters of the X-ray imaging system;
computing a noise level parameter, the computing of the noise level parameter comprising simulating an energy deposition of X-ray quanta emitted by an X-ray source of the X-ray imaging system on an X-ray detector of the X-ray imaging system depending on the set of imaging parameters; and
generating the noise-variance-stabilized X-ray image, the generating of the noise-variance-stabilized X-ray image comprising applying a variance-stabilizing transformation that depends on the noise level parameter to the X-ray image.

2. The computer-implemented method of claim 1, wherein the noise level parameter is computed depending on the simulated energy deposition by using a detector model for the X-ray detector that models a conversion of X-ray quanta to optical photons and a conversion of the optical photons to an electrical detector signal.

3. The computer-implemented method of claim 1, wherein the set of imaging parameters comprises:
a peak kilovoltage of the X-ray source;
a tube current of the X-ray source;
an X-ray pulse duration; or
any combination thereof.

4. The computer-implemented method of claim 1, wherein the set of imaging parameters comprises:
a filter material, filter thickness, or the filter material and the filter thickness of the X-ray filter of the X-ray imaging system;
a collimator opening size of an X-ray collimator of the X-ray imaging system; or
a combination thereof.

5. The computer-implemented method of claim 1, wherein the set of imaging parameters comprises:
a position, an orientation, or the position and the orientation of the X-ray source;
a position, an orientation, or the position and the orientation of the X-ray detector;
a position, an angulation, or the position and the angulation of a C-arm carrying the X-ray source and the X-ray detector;
a position, an orientation, or the position and the orientation of a patient table or a part of the patient table;
an effective thickness of an imaged object; or
any combination thereof.

6. The computer-implemented method of claim 1, wherein the set of imaging parameters comprises:
a gain factor of the X-ray detector;
a status parameter of an anti-scattering grid of the X-ray imaging system;
a pixel binning parameter of the X-ray detector; or
any combination thereof.

7. The computer-implemented method of claim 1, wherein the variance-stabilizing transformation is a generalized Anscombe transformation.

8. The computer-implemented method of claim 1, wherein a Monte Carlo simulation is used or a sequence of Monte Carlo simulations are used to simulate the energy deposition.

9. The computer-implemented method of claim 1, wherein simulating the energy deposition comprises:
simulating a collimation by an X-ray collimator of the X-ray imaging system;
simulating an X-ray attenuation, X-ray scattering, X-ray transmission, or any combination thereof by the imaged object, one or more further objects, or the imaged object and the one or more further objects.

10. A computer-implemented method for processing an X-ray image, the computer-implemented method comprising:
generating a noise-variance-stabilized X-ray image, the generating of the noise-variance-stabilized X-ray image comprising:
receiving the X-ray image generated by an X-ray imaging system and a corresponding set of imaging parameters of the X-ray imaging system;
computing a noise level parameter, the computing of the noise level parameter comprising simulating an energy deposition of X-ray quanta emitted by an X-ray source of the X-ray imaging system on an X-ray detector of the X-ray imaging system depending on the set of imaging parameters; and
generating the noise-variance-stabilized X-ray image, the generating of the noise-variance-stabilized X-ray image comprising applying a variance-stabilizing transformation that depends on the noise level parameter to the X-ray image; and
applying a denoising algorithm to the noise-variance-stabilized X-ray image; or
applying an image processing algorithm to the noise-variance-stabilized X-ray image.

11. The computer-implemented method of claim 10, wherein applying the denoising algorithm or the image processing algorithm comprises applying a trained machine learning model to the noise-variance-stabilized X-ray image).

12. An X-ray imaging method comprising:
generating, by an X-ray imaging system, an X-ray image;
processing the X-ray image, the processing of the X-ray image comprising carrying a computer-implemented method based on the X-ray image, the computer-implemented method comprising:
receiving the X-ray image generated by the X-ray imaging system and a corresponding set of imaging parameters of the X-ray imaging system;
computing a noise level parameter, the computing of the noise level parameter comprising simulating an energy deposition of X-ray quanta emitted by an X-ray source of the X-ray imaging system on an X-ray detector of the X-ray imaging system depending on the set of imaging parameters; and
generating a noise-variance-stabilized X-ray image, the generating of the noise-variance-stabilized X-ray image comprising applying a variance-stabilizing transformation that depends on the noise level parameter to the X-ray image; and
displaying, by a display device, the processed X-ray image or an image depending on the processed X-ray image.

13. A data processing system comprising:
a processor configured to generate a noise-variance-stabilized X-ray image, the processor being configured to generate the noise-variance-stabilized X-ray image comprising the processor being configured to:
receive an X-ray image generated by an X-ray imaging system and a corresponding set of imaging parameters of the X-ray imaging system;
compute a noise level parameter, the computation of the noise level parameter comprising simulation of an energy deposition of X-ray quanta emitted by an X-ray source of the X-ray imaging system on an X-ray detector of the X-ray imaging system depending on the set of imaging parameters; and
generate the noise-variance-stabilized X-ray image, the generation of the noise-variance-stabilized X-ray image comprising application of a variance-stabilizing transformation that depends on the noise level parameter to the X-ray image.

14. An X-ray imaging system comprising:
an X-ray source;
an X-ray detector;
a control system that is configured to control the X-ray source and the X-ray detector to generate an X-ray image;
a data processing system comprising a processor, the processor being configured to process the X-ray image, the processor being configured to process the X-ray image comprising the processor being configured to:
generate a noise-variance-stabilized X-ray image, the generation of the noise-variance-stabilized X-ray image comprising:
receipt of the X-ray image generated by the X-ray imaging system and a corresponding set of imaging parameters of the X-ray imaging system;
computation of a noise level parameter, the computation of the noise level parameter comprising simulation of an energy deposition of X-ray quanta emitted by the X-ray source of the X-ray imaging system on the X-ray detector of the X-ray imaging system depending on the set of imaging parameters; and
generation of the noise-variance-stabilized X-ray image, the generation of the noise-variance-stabilized X-ray image comprising application of a variance-stabilizing transformation that depends on the noise level parameter to the X-ray image; and
apply a denoising algorithm to the noise-variance-stabilized X-ray image; or
apply an image processing algorithm to the noise-variance-stabilized X-ray image; and
a display device,
wherein the control system is further configured to control the display device to display the processed X-ray image or an image depending on the processed X-ray image.
